(19) United States
(12) Patent Application Publication
(10) Pub. No.: US 2008/0160107 A1
(43) Pub. Date: Jul. 3, 2008

(54) Use of nitric oxide gas to treat blood and blood products

(75) Inventors: Frank J. McCaney, Voorhees, NJ (US); Alex Stenzler, Long Beach, CA (US); Chris Miller, North Vancouver (CA)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignees: Nitric Biotherapeutics, Inc., Bristol, PA (US); Cardinal Health 207, Inc., Yorba Linda, CA (US)

(21) Appl. No.: 11/982,260

(22) Filed: Oct. 31, 2007

Related U.S. Application Data
(63) Continuation-in-part of application No. 11/445,965, filed on Jun. 1, 2006.
Application No. 11/445,965 is a continuation-in-part of application No. 10/658,665, filed on Sep. 9, 2003.
(60) Provisional application No. 60/409,400, filed on Sep. 10, 2002.

(51) Int. Cl. A61K 33/00; B01J 19/00

(52) U.S. Cl. 424/718; 422/68.1

(57) ABSTRACT

The present invention relates to compositions and methods for treatment of blood and blood products using gaseous nitric oxide. The treatment involves the contacting blood or a blood product with gaseous nitric oxide.

### CROSS-REFERENCE TO RELATED APPLICATIONS

The present application is a Continuation-In-Part of U.S. patent application Ser. No. 11/445,965 filed on Jun. 1, 2006, which is a Continuation-In-Part of U.S. patent application Ser. No. 10/658,665, filed on Sep. 9, 2003, which is entitled to priority under 35 U.S.C. § 119(e) to U.S. Provisional Patent Application No. 60/409,400, filed on Sep. 10, 2002, each of which application is hereby incorporated herein by reference in its entirety.

### BACKGROUND OF THE INVENTION

Numerous techniques have been developed for the collection and storage of mammalian, and particularly, human blood outside of the body of a subject. The American Red Cross, for example, has developed methods for collecting and storing blood, and additional for using the stored blood. Stored blood is vital for the operating of healthcare systems worldwide, and is used for emergency transfusions, when a patient loses blood due to an accident or surgery.

As documented, however, transfusions using stored blood may have adverse effects, such as blocking capillaries, inducing tissue damage, and causing further vasoconstriction. Few studies have been conducted, but research to date suggests that the NO content of the stored blood may play a role in the decline in the usefulness of stored blood over time (Reynolds et al., (2007) PNAS, 104:17058-17062; Bennett-Guererro et al., (2007) PNAS, 104:17063-17068). In particular, preliminary evidence suggests that the addition of gNO to stored blood increases the utility of the stored blood in transfusions, and decreases the trauma and adverse effects resulting from blood transfusions.

Numerous techniques have also been developed for circulating the blood of a patient outside the body in an “extracorporeal” circuit and then returning it to the patient. For example, in dialysis for patients with kidney failure, blood is circulated extracorporeally and contacted with a large membrane surface separating the blood from a dialysate solution, and urea and other blood chemicals are migrated across the membrane to cleanse the blood, which is then returned to the patient. Another example of extracorporeal circulation is cardiopulmonary bypass (“CPB”), the procedure of mechanically bypassing both the heart and lungs to allow the whole heart to be isolated for surgical repair.

Several complications may arise in circulating blood outside of the patient's body. For example, contact of the blood with the foreign surfaces of the extracorporeal circuit triggers a massive defense reaction in blood proteins and cells that has been called “the whole body inflammatory response.” U.S. Pat. No. 5,957,880, herein incorporated by reference in its entirety, describes an improvement in extracorporeal circulation that employs contacting nitric oxide gas with the circulating blood. The nitric oxide gas was found to inhibit activation of blood platelets, thereby effecting a reduction or prevention of the whole body inflammation response heretofore associated with use of such apparatus.

In the 1980's, it was discovered by researchers that the endothelium tissue of the human body produced NO, and that NO is an endogenous vasodilator, namely, an agent that widens the internal diameter of blood vessels. Since this discovery, numerous medical applications of NO have developed. Researchers have discovered that inhaled NO may be used to treat various pulmonary diseases in patients. For example, NO has been investigated for the treatment of patients with increased airway resistance as a result of emphysema, chronic bronchitis, asthma, adult respiratory distress syndrome (ARDS), and chronic obstructive pulmonary disease (COPD).

U.S. Patent Publication No. 20040081580 describes a method for systematic delivery of the nitric oxide moiety in an extracorporeal circuit to reduce whole body contamination by pathogenic or toxic substances. Specific applications of the 20040081580 publication focus on managing bacteremia (blood poisoning) and/or septicemia in mammals. The 20040081580 publication describes the method of reducing pathogens in the mammal's blood stream to include the steps of: (1) providing an extracorporeal blood circuit; (2) circulating the mammal's blood through the extracorporeal blood circuit; and (3) exposing the blood in the circuit with nitric oxide gas in a concentration sufficient to reduce pathogenic content in the blood.

However, what is still needed in the art is a way to successfully treat extracorporeal blood and blood products, including products which are in a static state, and not necessarily in an active circuit, in order to improve the clinical outcome of blood transfusions. Accordingly, there is a need for a device and method for the extracorporeal treatment of blood and blood products using application of gaseous NO, in order to improve the safety and benefit of blood and blood product transfusions. The present invention meets these needs.

### BRIEF SUMMARY OF THE INVENTION

The invention includes a method of improving the outcome of transfusion in a patient in need thereof, comprising obtaining blood from a mammal, exposing blood to a NO-containing gas, increasing the level of NO in said blood as compared to the level of NO in the blood prior to the exposure, and administering the exposed blood to the patient, wherein the outcome of the transfusion is more favorable than the outcome of an otherwise identical transfusion conducted with blood that was not exposed to NO. In an aspect, a mammal and a patient according to the invention may or may not be the same organism.

In an aspect, the blood is stored blood. In another aspect, the blood is obtained from an extracorporeal circuit.

In the invention, a favorable outcome includes improvement of at least one of the outcomes selected from the group consisting of decreased inflammation, decreased vascular resistance, increased blood flow, and decreased tissue damage.

In an aspect, the invention includes a step of exposing the blood to oxygen, wherein exposure to oxygen occurs prior to transfusion.

The invention also includes a method of improving the outcome of transfusion in a patient in need thereof, comprising obtaining blood from a mammal, separating the blood into plasma and blood cells, exposing the plasma to a NO-containing gas, increasing the level of NO in said plasma as compared to the level of NO in the blood prior to exposure, combining the exposed plasma with blood cells, and administering the exposed blood to a patient, wherein the outcome of transfusion is more favorable than the outcome of an otherwise identical transfusion conducted with blood that was not exposed to NO.

In an aspect, the NO-containing gas is controllably introduced in relation to an amount of plasma separated from said blood.

In another aspect, the exposing step comprises providing a semipermeable membrane selectively permeable to NO gas and impermeable to nitrogen gas, adapted to allow contact of an outside of the membrane with said plasma; and delivering NO-containing gas to an inside of the membrane under pressure sufficient to drive the NO across the membrane for contact with the plasma on the outside of the membrane.

In an aspect, the concentration of NO in an NO-containing gas is about 1 ppm to about 200 ppm. In one aspect, the concentration of NO is about 20 ppm.

In an aspect of the invention, the concentration of NO in blood is measured at least one of the times selected from the group consisting of prior to exposure of blood to NO-containing gas and after exposure of blood to NO-containing gas. In an aspect, each measurement is selected from the group consisting of indirect measurement and direct measurement. In another aspect, the amount of NO to be added to blood is calculated based on the volume of blood to be exposed to NO. In another aspect, the amount of NO to be added to blood is calculated based on the amount of time that has elapsed since the blood was removed from a mammal. In another aspect, the amount of NO to be added to blood is calculated based on the amount of time remaining before the blood will be transfused into a patient.

In an aspect, a mammal is a human. In another aspect, a patient is a human.

In invention also includes a device for regulating the amount of gNO to be contacted with a sample of blood, comprising a probe for the detection of gNO and a mechanism for feedback regulation of the amount of gNO to be contacted with a sample of blood. In an aspect, the feedback regulation is based on at least one detected parameter and at least one user-defined parameter. In an aspect, a probe is capable of detecting gNO in a blood sample.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a flow chart of a method of reducing pathogens in blood, according to an embodiment of the invention.

FIG. 2 is a schematic of a diffusion conduit through which the nitric oxide containing gas may contact the plasma, according one embodiment of the invention.

FIG. 3 is a schematic showing the nitric oxide source and delivery.

### DETAILED DESCRIPTION

The present invention relates to compositions and methods for the treatment of blood and blood products outside of the body using gaseous nitric oxide (“NO”). The blood may be obtained from one or more individuals, treated according to the invention, and then administered to an unrelated individual, or to the same individual from whom the blood was obtained. This is because it is shown herein that gaseous NO (gNO) can be used to treat stored blood and blood products, in order to make the products safer and more beneficial for future use in mammals, including humans.

According to the present invention, transfusion of blood and blood products treated with gNO results in decreased inflammation and vascular resistance, as well as increased blood flow, resulting in decreased adverse reactions, including tissue damage.

### DEFINITIONS

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

As used herein, the term “modulate” is meant to refer to any change in biological state, i.e. increasing, decreasing, and the like.

As used herein, a “therapeutically effective amount” is the amount of a therapeutic composition sufficient to provide a beneficial effect to a mammal to which the composition is administered.

The terms “patient” and “individual” are interchangeably used to mean a warm-blooded animal, such as a mammal, who is the object of medical care. It is understood that humans and animals are included within the scope of the term “patient” or “individual.”

The term “treat” or “treatment,” as used herein, refers to the alleviation (i.e., “diminution”) and/or the elimination of a symptom or a source of a given disease.

“Evacuating” as the term is used herein, refers to the partial or complete removal of a substance from a specific region or area.

“Transfusion,” as the term is used herein, refers to the administration of blood or a blood product to the bloodstream of a patient. The term “transfusion” as used herein encompasses any method of administration to a patient, including intravenous administration from a container of stored blood, as well as re-administration of blood to a patient from an extracorporeal circuit.

“Stored blood” or a “stored blood product” is blood or a blood product which was obtained from a mammal, and is stored outside of the body of the mammal, in a container.

### Description

It is to be understood that this invention is not limited to the particular devices, compositions, methodologies or protocols described, as these may vary. It is also to be understood that the terminology used in the description is for the purpose of describing the particular versions or embodiments only, and is not intended to limit the scope of the present invention which will be limited only by the appended claims. Rather, the present invention encompasses any method or device that can be used to treat stored blood and stored blood products using gaseous nitric oxide.

Although any methods, devices, and materials similar or equivalent to those described herein can be used in the practice or testing of embodiments of the present invention, the preferred methods, devices, and materials are now described. All publications mentioned herein are incorporated by reference. Nothing herein is to be construed as an admission that the invention is not entitled to antedate such disclosure by virtue of prior invention. As used herein, terms such as “subject,” “patient,” and “mammal” may be used interchangeable. The mammal is preferably a human.

As set forth in detail elsewhere herein, stored blood tends to lose biological activity and effectiveness over time, with respect to the use of stored blood for transfusions. One aspect of blood that changes over time is the amount of NO bound to the hemoglobin in red blood cells (RBC). One way in which hemoglobin binds NO is through the beta-93 cysteine residue (Doctor et al. (2005) PNAA, 102:5709-5714). However, stored blood may also include NO in many other ways, in addition to the binding of NO by hemoglobin. NO may interact with other proteins, and a certain level of dissolved NO may exist in solution in stored blood.

The invention therefore provides methods of treating blood with gNO outside of the body, for the purpose of improving the outcome of blood transfusion in a patient in need thereof. In one aspect, stored blood is treated according to the invention. In another aspect, blood in an extracorporeal circuit is treated according to the invention. In either aspect of the invention, the blood is treated with gNO in order to improve the outcome of transfusion in a patient in need thereof. The outcome of transfusion is said to be improved when one or more clinical indicators associated with transfusion are improved. Such clinical indicators include, but are not limited to, decreased inflammation and vascular resistance, as well as to increase blood flow, resulting in decreased adverse reactions, including tissue damage, in the patient.

By way of a non-limiting example, the outcome of a transfusion in a patient is said to be improved when blood, treated according to the present invention, is administered to the patient, and as a result of the transfusion, the patient experiences increased blood flow, wherein the blood flow is more favorable than it would have been if the patient had been administered blood that was not treated with gNO.

It will be understood, based on the disclosure set forth herein, that the invention has additional applications, and encompasses any application in which the administration of NO to a patient can improve the process and/or outcome of transfusion. Other applications of the invention include, but are not limited to, transfusion of blood which has been contacted with gNO ex vivo, for the purposes of minimizing, decreasing or preventing the inflammatory response including endothelial damage, activation of blood components by a heart bypass apparatus, and ischemia with subsequent reperfusion injury.

Furthermore, many of the roles of NO in biology are well-characterized, and are known in the art, and will therefore not be reviewed extensively herein. The skilled artisan, when armed with the disclosure set forth herein, will understand which of these roles of NO are related to the outcome of the transfusion process, and therefore, which biological roles and/or effects of NO are effective therapeutic targets according to the present invention.

In an embodiment, the invention includes a method of increasing NO levels in extracorporeally-stored blood, wherein the NO levels in the stored blood were reduced from those levels of NO occurring naturally in a mammal. In an aspect, the invention includes injection of gNO into blood in an amount that restores the level of gNO to a level typically found naturally occurring in a mammal. In another aspect, the invention includes injection of gNO into blood in an amount that increases the level of gNO from the level existing prior to injection of gNO. In another aspect, the invention includes diffusion of gNO across a membrane that interfaces with the blood.

In an embodiment, the invention includes a method of measuring gNO to determine the level of NO prior to addition of gNO. In another embodiment, the invention includes a method of measuring gNO to determine the level of NO after the addition of gNO. In an aspect, the method is direct (e.g., direct detection of NO). In another aspect, the method is indirect (e.g., detection of a compound or reaction product which is indicative of the presence and/or concentration of NO). By way of a non-limiting example, gas phase NO may be measured, or blood serum NO may be measured.

In another embodiment of the invention, a method is included for calculating the amount of gNO to be added to the blood based on the volume of the blood to be contacted with gNO. In yet another embodiment, a method is included for calculating the amount of gNO to be added to the blood based on the time that the blood has been stored ex vivo prior to being contacted with gNO.

A device is also provided, the device comprising a probe for directly monitoring and/or measuring the gNO in blood. Such a device according to the invention may additionally comprise a mechanism for feedback control of the delivery of gNO to blood. It will be understood by the skilled artisan, when armed with the disclosure set forth herein, that the feedback of information and the regulation of gNO contact with blood can be adjusted according to the desired end result, as well as the starting conditions of the blood with respect to gNO, among other parameters.

The invention should not be considered to be limited by any particular method of contacting blood with gNO, or of delivering gNO to blood. Rather, the invention includes any method of providing gNO to blood, for use in any of the methods or compositions set forth herein. By way of a non-limiting example, a method of introducing gNO into blood comprises passing the blood and gNO through a device in which the partial pressure is kept constant, while the blood is contacted with the gNO. Under such conditions, gNO will readily diffuse in to the blood. Further, it will be understood that the gNO may be directly contacted with the blood, or in another embodiment, the gNO and blood may be introduced on opposite sides of a semi-permeable membrane or support.

In one aspect of the invention, gNO is added to blood just before re-transfusion into a patient. In another aspect, the skilled artisan may use the disclosure set forth herein to determine the optimal time to add gNO prior to re-transfusion. Therefore, gNO may also be added to blood well before re-transfusion into a patient. In one embodiment, whole blood is exposed to gNO. In another embodiment, plasma is exposed to gNO. In yet another embodiment, a blood product other than whole blood or plasma is exposed to gNO. I still another embodiment, a combination of at least two of whole blood, plasma or another blood product is exposed to gNO prior to re-transfusion into a patient.

The invention also includes a method of contacting blood with gNO in an extracorporeal circuit, as set forth elsewhere herein, in order to decrease inflammation and vascular resistance, as well as to increase blood flow, resulting in decreased adverse reactions, including tissue damage, in the patient, as the blood is recirculated back into the body of a patient.

Based on the length of time that blood is circulated outside of the body, or on the particular treatment to which the extracorporeal blood is subjected, among other things, the level of NO in the blood may decrease to a level below the normal or typical level found in the blood in the patient. Accordingly, addition of gNO to the blood in the extracorporeal circuit may be necessary, and may prevent one or more adverse effects associated with insufficient blood NO levels, as set forth in detail elsewhere herein.

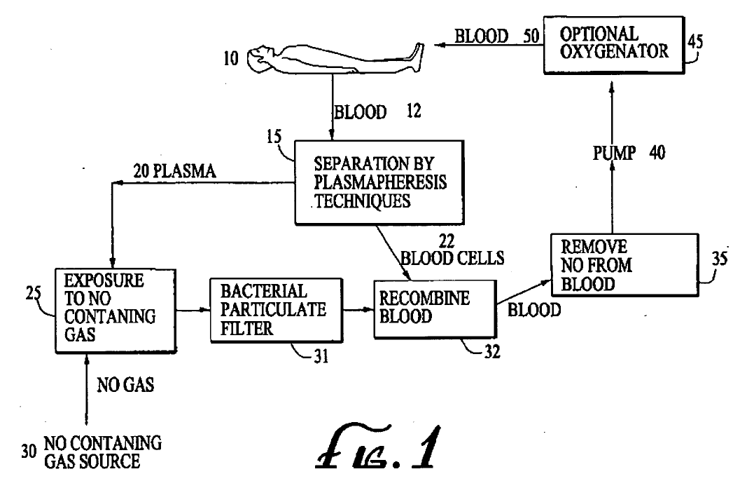

FIG. 1 represents a flow chart for a method of extracorporeal treatment of the blood. At step 12, blood is extracted from a patient 10 or blood source. For ease of description, the Applicants have focused on extraction from a human patient. However, the methods and devices are applicable to other mammals and may also be used to treat blood from any source, such as a blood bank. Any appropriate inlet line may be used to extract blood from a patient. For example, extraction may include inserting one or more venous catheter into the patient, either in a limb or central vein. Blood may be collected into an optional reservoir and then routed to the separator or blood may flow directly into the separator.

Next, at step 15, the extracted blood is separated into blood's two main components, i.e., the plasma or serum and the blood cells, including both red and white blood cells. This step may also be thought of as the removal of blood cells from the plasma. Several techniques may be used to separate blood into plasma 20 and blood cells 22. Such techniques may be borrowed from plasmapheresis techniques. Plasmapheresis is a blood purification procedure also known as plasma exchange. In plasmapheresis, blood is removed from a patient, blood cells are separated from plasma, fresh plasma is substituted for the extracted plasma, and the fresh plasma and blood cells are returned the patient. The present methods thus rely on the principles of separation exhibited in plasmapheresis techniques. These separation techniques include filtration, dialysis and centrifugation.

For example, in discontinuous flow centrifugation, about 300 mL of blood is centrifuged at a time to separate plasma from blood cells. In discontinuous flow, only one venous catheter line is required. Blood may be routed from the patient to a collection reservoir before batch configuration. A continuous flow centrifugation may also be practiced using two or more venous lines. This continuous procedure requires slightly less blood volume to be out of the patient at any one time. In plasma filtration, two venous line are used. The plasma is filtered out of the blood using standard hemodialysis equipment. Less than 100 mL of blood are required to be outside the patient at one time using this filtering technique.

Once plasma has been isolated from the blood, it may be exposed to nitric oxide containing gas, at step 25. As described in the background section, nitric oxide gas has been used against pathogens, such as viruses, bacteria, mycobateria, parasites, and fungi. These pathogens, if blood borne, may be found in the patient's plasma or serum. To more effectively target the destruction of pathogens in a patient's blood, the isolated plasma is exposed to nitric oxide containing gas. This direct exposure of the plasma to a nitric oxide containing gas, as compared to blood (plasma and blood cells) is a highly effective decontamination technique.

At step 25, exposing the plasma to a nitric oxide containing gas may be accomplished using the techniques described in the parent application, U.S. Patent Publication No. 20040081580, herein incorporated in its entirety. The NO-containing gas may be supplied at step 30. Appropriate techniques for diluting NO gas to usable concentrations may be employed, such as appropriate blending of pure NO with other carrier gases. Carrier gases may include air, nitrogen, and oxygen. The methods of the present invention may use a NO-containing gas having a NO concentration of about 1 ppm to about 200 ppm, including any and all integers including and in between 1 ppm and 200 ppm. In an embodiment of the invention, the concentration of NO is 20 ppm.

However, it will be understood that, based on the disclosure set forth herein, the skilled artisan will understand how to adjust the concentration of gNO used to contact blood, based on the starting conditions and on the desired outcome of the exposure of blood to gNO. By way of several non-limiting examples, the concentration of gNO may be adjusted, or increased beyond 200 ppm for contacting a large volume of blood, the concentration of gNO may be adjusted, or increased beyond 200 ppm for contacting a volume of blood for a brief period of time, the concentration of gNO may be adjusted, or increased beyond 200 ppm for contacting a large volume of blood for a brief period of time, and the concentration of gNO may be adjusted for a different desired outcome of the exposure of blood to gNO.

To restore gNO to physiologically normal levels, amount of gNO added is dependent on a number of factors, including the volume of blood or plasma being treated, the length of time the blood has been stored, and the time remaining until transfusion of the blood into a patient. Blood that is added to the body with decreased gNO levels may lead to vasoconstriction in the arterio-capillary bed and lower than normal levels of oxygenation. Adding gNO to blood prior to transfusion may restore normal levels and increase oxygenation to the tissue. Patients in need of large transfusions are the most in need of sufficient oxygenation and the most critical for normal, physiological levels of gNO.

In an aspect of the invention, gNO is added to blood to restore physiological levels of gNO. In another aspect, gNO is added to blood to provide supra-physiological levels of gNO.

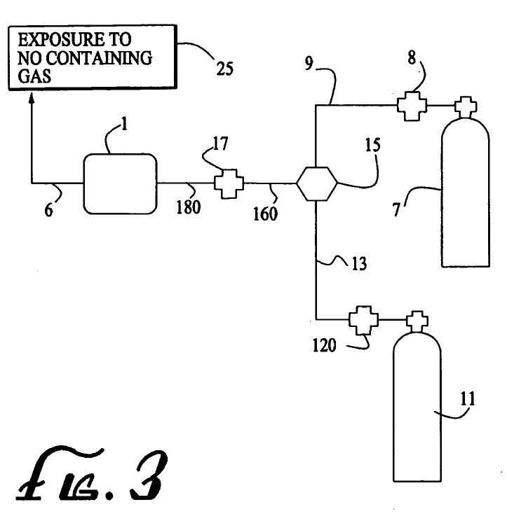

The nitric oxide containing gas may be dosed and delivered using known delivery techniques. See FIG. 3, wherein a schematic is shown demonstrating one manner of delivery of NO gas. The NO source 7, can be a pressurized cylinder containing NO gas, and a nitric oxide flow control valve/pressure regulator 8, delivering NO to the gaseous nitric oxide delivery device 1 through supply tubing 9 and an optional gas blender 15. In FIG. 3, the NO gas source 7 is a pressurized cylinder containing NO gas. While the use of a pressurized cylinder is the preferable method of storing the NO containing gas source 7, other storage and delivery means, such as a dedicated feed line can also be used. Typically the NO gas source 7 is a mixture of N₂ and NO. While N₂ is typically used to dilute the concentration of NO within the pressurized cylinder, any inert gas can also be used.

When the NO gas source 7 is stored in a pressurized cylinder, it is preferable that the concentration of NO in the pressurized cylinder fall within the range of about 800 ppm to about 1200 ppm. Commercial nitric oxide manufacturers typically produce nitric oxide mixtures for medical use at around the 1000 ppm range. Extremely high concentrations of NO are undesirable because accidental leakage of NO gas is more hazardous, and high partial pressures of NO tends to cause the spontaneous degradation of NO into nitrogen. Pressurized cylinders containing low concentrations of NO (i.e., less than 100 ppm NO) can also be used in accordance the device and method disclosed herein. Of course, the lower the concentration of NO used, the more often the pressurized cylinders will need replacement.

FIG. 3 also shows source of diluent gas 11 as part of the NO delivery device 1 that is used to dilute the concentration of NO for delivery to the gaseous NO delivery device 1 through line 13. The source of diluent gas 11 can contain N₂, O₂, air, an inert gas, or a mixture of these gases. It is preferable to use a gas such as N₂ or an inert gas to dilute the NO concentration since these gases will not oxidize the NO into NO₂, as would O₂ or air. The source of diluent gas 11 is shown as being stored within a pressurized cylinder. While the use of a pressurized cylinder is shown in FIG. 3 as the means for storing the source of diluent gas 11, other storage and delivery means, such as a dedicated feed line can also be used. The NO gas from the NO gas source 7 and the diluent gas from the diluent gas source 11 preferably pass through flow control valve/pressure regulators 8, 120, to reduce the pressure of gas that is admitted to the gaseous NO delivery device 1.

The respective gas streams pass via tubing 9, 13, to an optional gas blender 15. The gas blender 15 mixes the NO gas and the diluent gas to produce a NO-containing gas that has a reduced concentration of NO. Preferably, the NO-containing gas that is output from the gas blender 15 has a concentration that is greater than about 100 ppm. The NO-containing gas that is output from the gas blender 15 travels via tubing 160 to a flow control valve 17. The flow control valve 17 can include, for example, a proportional control valve that opens (or closes) in a progressively increasing (or decreasing if closing) manner. As another example, the flow control valve 17 can include a mass flow controller. The flow control valve 17 controls the flow rate of the NO-containing gas that is input to the gaseous NO delivery device 1. The NO-containing gas leaves the flow control valve 17 via flexible tubing 180. The flexible tubing 180 attaches to an inlet of the gaseous NO delivery device 1. The inlet for 1 might include an optional one-way valve that prevents the backflow of gas. From flexible tubing 6, the NO containing gas is routed to unit 25 (FIG. 1), wherein the plasma is exposed to the gas.

An effective amount, i.e., an amount sufficient to reduce the content of pathogens in the plasma, is generally greater than about 100 ppm nitric oxide gas. A flowrate of about 1 liter per minute of about 160 ppm nitric oxide to about 400 ppm nitric oxide may be delivered to the exposure unit. The nitric oxide containing gas is controllably delivered in relation to the amount of plasma being treated.

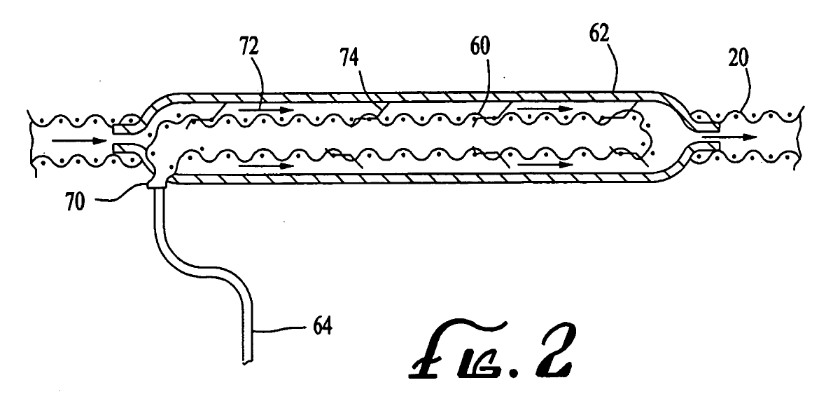

A semipermeable membrane selectively permeable to nitric oxide gas and impermeable to nitrogen gas may provide an effective exposure technique at step 25 (FIG. 1). The outside of the membrane contacts the plasma, while the inside of the membrane provides the interface for the nitric oxide containing gas. The nitric oxide containing gas is delivered to the inside of the membrane under pressure sufficient to drive the nitric oxide across the membrane, contacting the plasma of the other side. Such contact may be accomplished with the diffusion device illustrated in FIG. 2. In an aspect, such a membrane may also comprise a NO detector.

Referring to FIG. 2, the gas permeable membrane 60 is elongated and tubular in form and is disposed longitudinally within conduit 62 adapted to come into contact with plasma flowing through the diffusion conduit 62. The nitric oxide containing gas is supplied through tubing 64 and flows into the interior of gas permeable membrane 60. Due to the permeability of this membrane 60 to nitric oxide gas, the gas will diffuse through the membrane and dissolve in the plasma where it will come in contact with pathogens. The membrane 60 is selected to be impermeable to the carrier gas, such as nitrogen or air and thus the carrier gas will not diffuse through the membrane. The nitric oxide containing gas flows into the membrane 60 at location 70. As the gas pressure inside the gas permeable membrane 60 exceeds the pressure of the plasma within conduit 62, nitric oxide gas will diffuse from the membrane into the plasma as indicated by arrows 74. The plasma flows through the diffusion conduit 62 as illustrated by arrows 72.

Before recombining the treated plasma and the blood cells at step 32, the treated plasma may optionally be run through a bacterial particulate filter to remove lipopolysaccharide (LPS) material, at step 31. LPS is a result of dead bacteria as their cell walls are made up of this material. Excessive levels of LPS may cause an inflammatory response once the recombined blood is returned to the body, even if the bacteria in the plasma are dead. The line before the filter step 31 may also have a LPS monitor (not illustrated) to determine if the removal through filter step 31 is necessary. Thus, LPS is preferably removed before combining the treated plasma with the blood cells.

At step 32, the treated plasma and the blood cells are recombined in any suitable manner. Plasmapheresis techniques of recombining plasma and blood cells may be specifically employed. Therefore, the blood after the recombining step 32 contains dissolved nitric oxide gas.

The extracorporeal circuitry may include one or more pumps 40 necessary to transport the blood from one step to the next, before return to the patient. Additionally illustrated in FIG. 1 at step 45 is an optional oxygenator, such as the one described in U.S. Patent Publication No. 20040081580 used to expose the blood to oxygen gas. The oxygenator may treat the blood before it has been separated into the plasma and blood cells. Alternatively, the oxygenator may be downstream from the separation unit, such as located after the recombination of the treated plasma and the blood cells.

The extracorporeal circuitry may include: (1) an inlet line adapted to receive blood from a mammal or a blood source; (2) an outlet line adapted to return blood to the mammal or blood source; and (3) a fluid circuit for fluid communication between the inlet and the outlet line. Other components of the fluid circuit include: (1) at least one pump to circulate the blood; (2) a separation unit in fluid communication with the inlet line, wherein the separation unit is adapted to separate the blood received from the mammal or source into plasma and blood cells; (3) a nitric oxide unit that exposes the plasma with a nitric oxide gas containing gas; and (4) a mixer for combining the exposed plasma with the blood cells.

Several optional components may be included into the circuitry. For example, a reservoir may be used to collect the blood from the mammal or source and thus monitor the amount of blood entering the separation unit. Additionally, in accordance with traditional uses of extracorporeal equipment and procedures, an oxygenator, a dialysis component, an organ perfusion component, a heat exchange component, and/or an oxygenation component may be incorporated into the circuitry. Such devices are known in the art. Optionally, blood circulating through the circuitry may be treated with an anticlotting agent to prevent clotting. Furthermore, the circuitry includes the necessary flexible tubing and pump devices for circulating the fluids.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of improving the outcome of transfusion in a patient in need thereof, said method comprising:
a. obtaining blood from a mammal,
b. exposing said blood to a NO-containing gas, increasing the level of NO in said blood as compared to the level of NO in said blood prior to said exposing, and
c. administering said exposed blood to said patient, wherein the outcome of said transfusion is more favorable than the outcome of an otherwise identical transfusion conducted with blood that was not exposed to NO, further wherein said mammal and said patient may or may not be the same organism.

2. The method of claim 1, wherein said blood is stored blood.

3. The method of claim 1, wherein said blood is obtained from an extracorporeal circuit.

4. The method of claim 1, wherein said favorable outcome comprises improvement of at least one of the outcomes selected from the group consisting of decreased inflammation, decreased vascular resistance, increased blood flow, and decreased tissue damage.

5. A method of improving the outcome of transfusion in a patient in need thereof, said method comprising:
a. obtaining blood from a mammal,
b. separating the blood into plasma and blood cells,
c. exposing said plasma to a NO-containing gas, increasing the level of NO in said plasma as compared to the level of NO in said blood prior to said exposing,
d. combining said exposed plasma with said blood cells, and
e. administering said exposed blood to said patient,
wherein the outcome of said transfusion is more favorable than the outcome of an otherwise identical transfusion conducted with blood that was not exposed to NO, further wherein said mammal and said patient may or may not be the same organism.

6. The method of claim 1, further comprising the step of exposing said blood to oxygen, wherein said exposing to oxygen step occurs prior to step (c).

7. The method of claim 5, wherein said NO-containing gas is controllably introduced in relation to an amount of plasma separated from said blood.

8. The method of claim 5, wherein said exposing step comprises: providing a semipermeable membrane selectively permeable to NO gas and impermeable to nitrogen gas, adapted to allow contact of an outside of the membrane with said plasma; and delivering said NO-containing gas to an inside of said membrane under pressure sufficient to drive said NO across said membrane for contact with said plasma on the outside of said membrane.

9. The method of claim 1, wherein the concentration of NO in said NO-containing gas is about 1 ppm to about 200 ppm.

10. The method of claim 9, wherein said concentration of NO is about 20 ppm.

11. The method of claim 1, wherein the concentration of said NO in said blood is measured at least one of the times selected from the group consisting of prior to said exposure of said blood to said NO-containing gas and after said exposure of said blood to said NO-containing gas.

12. The method of claim 11, wherein each of said measurement is selected from the group consisting of indirect measurement and direct measurement.

13. The method of claim 1, further wherein the amount of said NO to be added to said blood is calculated based on the volume of blood to be exposed to said NO.

14. The method of claim 1, further wherein the amount of said NO to be added to said blood is calculated based on the amount of time that has elapsed since said blood was removed from said mammal.

15. The method of claim 1, further wherein the amount of said NO to be added to said blood is calculated based on the amount of time remaining until said blood will be transfused into said patient.

16. The method of claim 1, wherein said mammal is a human, further wherein said patient is a human.

17. A device for regulating the amount of gNO to be contacted with a sample of blood, said device comprising a probe for the detection of gNO and a mechanism for feedback regulation of the amount of gNO to be contacted with a sample of blood, wherein said feedback regulation is based on at least one detected parameter and at least one user-defined parameter.

18. The device of claim 17, wherein said probe is capable of detecting gNO in a blood sample.
